(19) United States
(12) Patent Application Publication
(10) Pub. No.: US 2026/0157867 A1
(43) Pub. Date: Jun. 11, 2026

(54) STENT WITH IMPROVED ANTI-MIGRATION PROPERTIES

(71) Applicant: BOSTON SCIENTIFIC SCIMED, INC., Maple Grove, MN (US)

(72) Inventor: Martyn G. Folan, Loughrea (IE)

(73) Assignee: BOSTON SCIENTIFIC SCIMED, INC., Maple Grove, MN (US)

(21) Appl. No.: 19/180,860

(22) Filed: Apr. 16, 2025

Related U.S. Application Data
(60) Provisional application No. 63/635,196, filed on Apr. 17, 2024.

(51) Int. Cl. A61F 2/848; A61F 2/90

(52) U.S. Cl. CPC A61F 2/848; A61F 2/90; A61F 2220/0008; A61F 2230/0006; A61F 2230/0069; A61F 2230/0091; A61F 2230/0095; A61F 2250/0039

(57) ABSTRACT

A self-expanding stent configured to shift between a delivery configuration and a deployed configuration, the self-expanding stent including a tubular body having a proximal end and a distal end. In embodiments, the tubular body of the self-expanding stent defines a proximal flange, a distal flange, and a saddle portion extending from the proximal flange to the distal flange. The proximal flange and/or the distal flange may include an open-cell foam material affixed adjacent to or within the proximal flange and/or the distal flange to promote tissue ingrowth therein. Additional flanges, flange orientations, materials, and disposition of materials for a self-expanding stent are also contemplated.

### CROSS-REFERENCE TO RELATED APPLICATIONS

The application claims the benefit of U.S. Provisional Patent Application Ser. No. 63/635,196 filed on Apr. 17, 2024, the disclosure of which is incorporated herein by reference.

### TECHNICAL FIELD

The present disclosure pertains to medical devices, and methods for using and employing medical devices. More particularly, the present disclosure pertains to an improved design and method of use for an endoprosthesis or stent.

### BACKGROUND

Some conditions may cause body lumens (e.g., the esophagus, the bile duct, the trachea, the gastrointestinal tract, the vascular system, etc.) to become restricted, such as by a stricture formation. As a result, it may be necessary to open the body lumen to permit normal function. Some body lumens may be treated with a self-expanding stent. However, some body lumens are also prone to unintended and/or undesired movement or migration of the stent. Some stents have been designed with a flare on one or both ends with an intent to improve anchoring within the body lumen. However, the flared end(s) can sometimes have undesirable consequences, including perforation of the body lumen and/or stenosis formation. There is an ongoing and unmet need to provide alternative endoprostheses or stents as well as alternative methods for using and employing endoprostheses or stents.

### SUMMARY

In a first aspect, a self-expanding stent, such as an esophageal stent, may comprise a tubular body configured to shift between a delivery configuration and a deployed configuration, the tubular body having a proximal end and a distal end and formed of one or more interwoven elements. In either configuration, the tubular body may define a proximal flange near the proximal end of the tubular body. The tubular body may further include a saddle region extending longitudinally and distally from the proximal flange. The proximal flange may further include an open-cell foam material affixed within or adjacent to the proximal flange. The open-cell foam material can promote and allow tissue ingrowth within the open-cell foam material, thereby further counteracting migration of the self-expanding stent of the present disclosure.

In addition or alternatively, the tubular body of the self-expanding stent may further include a distal flange disposed and/or situated near the tubular body distal end.

In addition or alternatively, the tubular body of the self-expanding stent may further include a distal support flange disposed and/or situated proximally to and near the distal flange.

In addition or alternatively, the tubular body of the self-expanding stent may further include a proximal support flange disposed and/or situated distally to and near the proximal flange.

In addition or alternatively, a space between the proximal flange and the proximal support flange of the tubular body may define a “proximal inter-flange space.” The proximal inter-flange space may further include an open-cell foam material disposed within or affixed within or adjacent to the proximal inter-flange space.

In addition or alternatively, the open-cell foam material may extend helically or longitudinally from the proximal flange and along the tubular body.

In addition or alternatively, the open-cell foam material may be interspersed with or within the proximal flange, such that at least one or more portions of the proximal flange and at least one or more portions of open-cell foam material is visible from outside the self-expanding stent.

In addition or alternatively, the open-cell foam material may be ring-shaped or annular.

In addition or alternatively, at least one or more flanges of the tubular body may project from the tubular body at an angle ranging between 30 to 60 degrees with respect to the longitudinal axis of the tubular body.

In addition or alternatively, a self-expanding stent of the present disclosure may include a tubular body having a proximal end and a distal end. The tubular body may further be formed of one or more interwoven filaments. The tubular body may further include a proximal flange near the proximal end of the tubular body and a distal flange near the distal end of the tubular body. One or more of the proximal flange and the distal flange may project from the tubular body at an angle ranging between 30 to 60 degrees. Further, the tubular body may also include a saddle region, the saddle region extending between the proximal flange and the distal flange.

In addition or alternatively, the tubular body may further include a proximal support flange situated distally to and near the proximal flange. Further, the tubular body may also include a distal support flange situated proximally to and near the distal flange.

In addition or alternatively, at least one or more of the proximal support flange and the distal support flange of the tubular body may project from the tubular body at an angle ranging between 30 to 60 degrees with respect to the longitudinal axis of the tubular body.

In addition or alternatively, methods of deploying a self-expanding stent within a body lumen of a patient are also contemplated by the present disclosure. An example non-limiting method includes a process of deploying and implanting a self-expanding stent within a body lumen of a patient, the method further including an implanting step. The implanting step of this non-limiting example method further includes radially expanding a proximal flange of the self-expanding stent against a wall of the body lumen. The implanting step further includes radially expanding a distal flange of the self-expanding stent against a wall of the body lumen, such that a saddle region of the self-expanding stent extends between the proximal flange and the distal flange of the self-expanding stent. The method further includes an open-cell foam material positioned on the stent adjacent to at least one of the proximal flange and the distal flange for promoting and allowing tissue ingrowth within the open-cell foam material.

In addition or alternatively, exemplary methods also include a self-expanding stent incorporating a proximal flange with a first diameter and a distal flange with a second diameter. In some instances, the first diameter is a different diameter than the second diameter.

The above summary of some embodiments, aspects, and/or examples is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 illustrates aspects of an example stent of the present disclosure;

FIG. 2 illustrates further aspects of an example stent of the present disclosure;

FIG. 3 illustrates yet further aspects of an example stent of the present disclosure; and

FIGS. 4A-4C illustrate aspects of example stents of the present disclosure, including various implementations of an open-cell foam material;

FIGS. 5A and 5B illustrate further aspects of example stents of the present disclosure, including various implementations of an open-cell foam material;

FIG. 6 illustrates additional aspects of an example stent of the present disclosure, including flanges that are angled with respect to the tubular body of an example stent of the present disclosure;

FIG. 7 illustrates additional aspects of an example stent of the present disclosure, including multiple flanges that are angled with respect to the tubular body of an example stent of the present disclosure;

FIG. 8A illustrates additional aspects of an example stent of the present disclosure, including multiple flanges that include a grooved flange construction.

FIG. 8B illustrates additional aspects of an example stent of the present disclosure, including multiple flanges that include a split-flange construction.

FIG. 9 illustrates additional aspects of forming an example stent of the present disclosure, incorporating the usage of a mandrel.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the claimed invention. Those skilled in the art will recognize that the various elements described and/or shown may be arranged in various combinations and configurations without departing from the scope of the disclosure. The detailed description and drawings illustrate example embodiments of the claimed invention.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the disclosed invention are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The term “extent” may be understood to mean a greatest measurement of a stated or identified dimension, unless the extent or dimension in question is preceded by or identified as a “minimum”, which may be understood to mean a smallest measurement of the stated or identified dimension. For example, “outer extent” may be understood to mean an outer dimension, “radial extent” may be understood to mean a radial dimension, “longitudinal extent” may be understood to mean a longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently—such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete structures or elements together.

The terms “annular” or “annulus” shall generally refer to an element or structure having a rounded, ring, or ringed shape.

The term “tissue ingrowth” shall generally refer to the phenomenon where outside tissue expands into and binds within the framework of an implanted stent or endoprosthesis, creating a hold on the stent with the ingrown tissue and thereby increasing the difficulty of stent migration.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to implement the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The figures illustrate selected components and/or arrangements of an endoprosthesis or stent. It should be noted that in any given figure, some features of the endoprosthesis or stent may not be shown, or may be shown schematically, for simplicity. Additional details regarding some of the components of the endoprosthesis or stent may be illustrated in other figures in greater detail. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For example, a reference to “the flange”, “the end”, “the filament”, or other features may be equally referred to all instances and quantities beyond one of said feature. As such, it will be understood that the following discussion may apply equally to any and/or all of the components for which there are more than one within the endoprosthesis or stent, unless explicitly stated to the contrary. Additionally, not all instances of some elements or features may be shown in each figure for clarity.

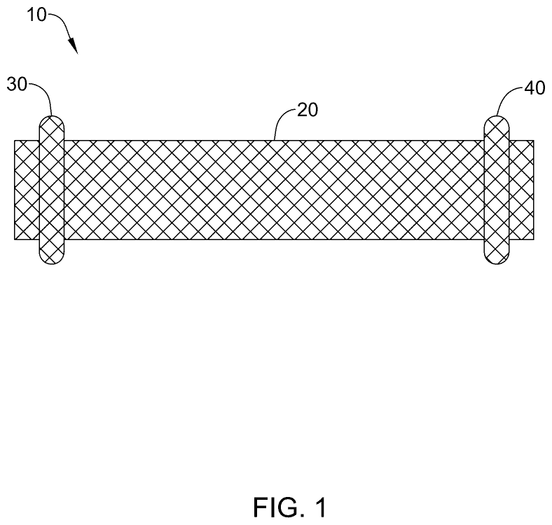

FIG. 1 illustrates an example self-expanding stent 10 (which term may be used interchangeably with the term “stent” and “endoprosthesis”) comprising a tubular body configured to shift between a delivery configuration (e.g., a radially constrained delivery configuration) and a deployed configuration (e.g., radially expanded delivery configuration), the tubular body of the self-expanding stent 10 having a proximal flange 30 and/or a distal flange 40 with a saddle region 20 (e.g., tubular body region) extending between the proximal flange 30 and the distal flange 40. In some aspects, the delivery configuration may be axially elongated and/or radially collapsed or compressed compared to the deployed configuration. The deployed configuration may be axially shortened and/or radially expanded compared to the delivery configuration.

The proximal flange 30, distal flange 40 and any other additional flanges contemplated by this disclosure may serve myriad purposes. For instance, the proximal flange 30, distal flange 40, and any other additional flanges contemplated by this disclosure may provide stent retention and/or impede stent migration without causing undue trauma or damage to patient tissue (i.e., lumen, organ or blood vessel walls) contacted by the proximal flange, distal flange, and any other additional flanges contemplated by the present disclosure.

In some embodiments, the tubular body of the self-expanding stent 10 may comprise an expandable framework. In some instances, the expandable framework may be formed of one or more, or a plurality of interwoven wires or filaments. For example, the expandable framework may be formed of a plurality of braided wires or filaments in some instances. The expandable framework may also be equivalently defined and/or categorized as “stent scaffolding” or “stent material”. The one or more, or plurality of interwoven wires or filaments may extend throughout the length of the tubular body including the proximal flange 30, the distal flange 40, and the saddle region 20. In some instances, the one or more, or plurality of interwoven wires or filaments may extend continuously along an entire length of the tubular body of the stent 10 such that the one or more, or plurality of interwoven wires or filaments may extend continuously throughout the proximal flange 30, the distal flange 40, and the saddle region 20 extending therebetween.

The proximal flange 30 and/or the distal flange 40 may be considered a double wall flange in which the one or more, or plurality of interwoven wires or filaments extend radially outward from the saddle region 20 away from the central longitudinal axis of the stent 10 (forming a first wall of the double wall flange) and then turn back toward the central longitudinal axis of the stent 10 (forming a second wall of the double wall flange). An apex or outermost extent of the proximal flange 30 may be located between the first and second radially extending walls of the proximal flange 30. Likewise, an apex or outermost extent of the distal flange 40 may be located between the first and second radially extending walls of the distal flange 40.

In at least some embodiments, the tubular body and/or the expandable framework may be self-expandable. For example, the tubular body and/or the expandable framework of the self-expanding stent 10 may be formed from a shape memory material. In some embodiments, the tubular body and/or the expandable framework may be mechanically expandable. For example, the tubular body and/or the expandable framework may be expandable using an inflatable balloon, using an actuation member, pneumatically or by other suitable or equivalent means. During delivery to a treatment site, the tubular body and/or the expandable framework of the self-expanding stent 10 may be disposed within a lumen of a delivery sheath in the delivery configuration. Upon removal from the lumen of the delivery sheath, the tubular body of the self-expanding stent 10 and/or the expandable framework may be shifted to the deployed configuration.

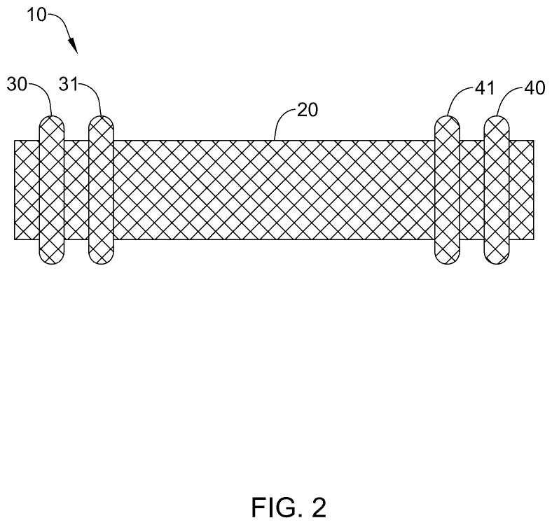

In some aspects, for example the configuration shown in FIG. 2, the tubular body and/or the expandable framework of the self-expanding stent 10 may define a proximal flange 30 disposed near the proximal end of the tubular body of self-expanding stent 10, a distal flange 40 disposed near the distal end of the tubular body of the self-expanding stent 10, a proximal support flange 31 disposed distally to and near the proximal flange, a distal support flange 41 disposed proximally to and near the distal flange, and a saddle region 20 extending between the proximal support flange 31 and the distal support flange 41. In other words, the proximal end region of the stent 10 may include a proximal flange 30 and a proximal support flange 31 proximate thereto with an annular gap therebetween, and/or the distal end region of the stent 10 may include a distal flange 40 and a distal support flange 41 proximate thereto with an annular gap therebetween.

The proximal support flange 31 and/or the distal support flange 41 may be formed substantially similar to the proximal flange 30 and/or the distal flange 40. In at least some embodiments, the proximal flange 30, the proximal support flange 31, the distal flange 40 and the distal support flange 41 may be continuously formed with the saddle region 20 as a single continuous structure. For instance, the one or more, or plurality of interwoven filaments forming the saddle region 20 may extend proximal of the saddle region 20 throughout the proximal support flange 31 and the proximal flange 30 to form the proximal support flange 31 and the proximal flange 30, and/or the one or more, or plurality of interwoven filaments forming the saddle region 20 may extend distal of the saddle region 20 throughout the distal support flange 41 and the distal flange 40 to form the distal support flange 41 and the distal flange 40. Additionally or alternatively, one or more of the proximal flange 30, the proximal support flange 31, the distal flange 40 and the distal support flange 41 may be affixed to or coupled with the tubular body of the self-expanding stent 10 as separate elements. Thus in some instances, the one or more, or plurality of interwoven filaments forming the saddle region 20 may extend along an entire length of the stent 10 and form each of the proximal flange 30, the proximal support flange 31, the saddle region 20, the distal support flange 41, and the distal flange 40.

The proximal flange 30, proximal support flange 31, distal flange 40, distal support flange 41 and any other additional flanges contemplated by this disclosure may serve myriad purposes. For instance, the proximal flange 30, proximal support flange 31, distal flange 40, distal support flange 41 and any other additional flanges contemplated by this disclosure may provide stent retention in a body lumen or across body structures and/or impede stent migration without causing undue trauma or damage to patient tissue (i.e., lumen, organ or blood vessel walls) contacted by the proximal flange, proximal support flange, distal flange, distal support flange and any other additional flanges contemplated by the present disclosure.

The spacing between flanges of the present disclosure may be variable and may ascribe to myriad suitable ranges. For instance, the spacing between the proximal flange 30 and the distal flange 40 may be about 20 mm or more. In other instances, the spacing between the proximal flange 30 and the distal flange 40 may be about 30 mm or more. In yet other instances, the spacing between the proximal flange 30 and the distal flange 40 may be about 10 mm or more, although various distances and ranges of distances are contemplated, for example a range of distances between 5 and 100 mm between the proximal flange 30 and the distal flange 40 is contemplated by the present disclosure.

Alternatively or additionally, the spacing between the proximal support flange 31 and the proximal flange 30 may be about 0.5 mm. In yet other aspects, the spacing between the proximal support flange 31 and the proximal flange 30 may be about 2 mm. In yet further aspects, the spacing between the proximal support flange 31 and the proximal flange 30 may be about 5 mm. In some instances, the spacing between the proximal support flange 31 and the proximal flange 30 may be in the range of about 0.5 mm to about 10 mm, in the range of about 0.5 mm to about 5 mm, in the range of about 0.5 mm to about 2 mm, or in the range of about 1 mm to about 2 mm. A wider range of distances is further contemplated, including but not limited to a range of distances spanning between 0.01 mm and 10 mm.

Alternatively or additionally, the spacing between the distal support flange 41 and the distal flange 40 may be about 0.5 mm. In yet other aspects, the spacing between the distal support flange 41 and the distal flange 40 may be about 2 mm. In yet further aspects, the spacing between the distal support flange 41 and the distal flange 40 may be about 5 mm. In some instances, the spacing between the distal support flange 41 and the distal flange 40 may be in the range of about 0.5 mm to about 10 mm, in the range of about 0.5 mm to about 5 mm, in the range of about 0.5 mm to about 2 mm, or in the range of about 1 mm to about 2 mm. A wider range of distances is further contemplated, including but not limited to a range of distances spanning between 0.01 mm and 10 mm.

Alternatively or additionally, the spacing between the distal support flange 41 and the proximal support flange 31, and thus the length of the saddle region 20, may be about 5 mm or more. In yet other aspects, the spacing between the distal support flange 41 and the proximal support flange 31 may be about 10 mm or more. In yet further aspects, the spacing between the distal support flange 41 and the proximal support flange 31 may be about 15 mm or more, although a range of distances is contemplated, including but not limited to a range of distances spanning between 5 mm and 100 mm.

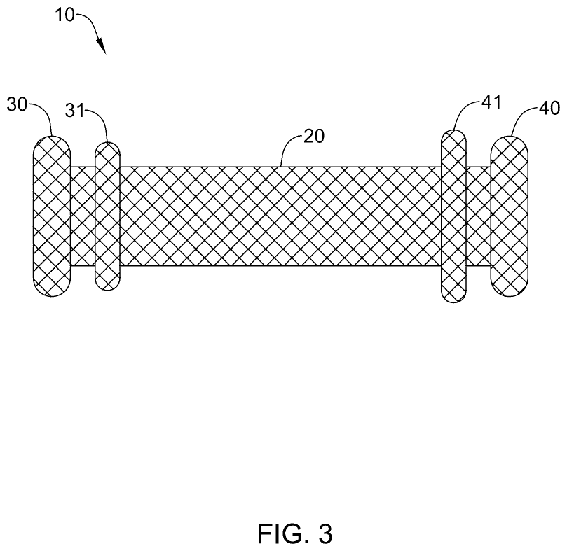

FIG. 3 shows another aspect of the present disclosure. In this aspect, a self-expanding stent 10 is depicted including a saddle region 20, a proximal flange 30, a proximal support flange 31, a distal flange 40, and a distal support flange 41. As shown in FIG. 3, the saddle region 20 extends between the proximal support flange 31 and the distal support flange 41, with the proximal flange 30 located proximal of the proximal support flange 31 and the distal flange 40 located distal of the distal support flange 41.

Myriad lengths of the saddle region 20 (i.e., saddle portion) are contemplated. For instance, in certain embodiments the saddle region 20 may be of a length of about 20 mm or more. In other embodiments, the saddle region 20 may be of a length of about 10 mm or more. In yet other embodiments, the saddle region 20 may be of a length of about 30 mm or more. In further instances, the saddle region 20 may be of a length in the range of about 10 mm to about 50 mm. In yet other instances, the saddle region 20 may be of a length in the range of about 10 mm to about 100 mm. In additional instances, the saddle region 20 may be of a length in the range of about 10 to about 200 mm. Various other dimensions other than the aforementioned ranges is contemplated as a possible length for the saddle region 20 of the present disclosure.

The proximal flange 30, proximal support flange 31, distal flange 40, distal support flange 41 and any other additional flanges contemplated by the present disclosure may be of myriad widths (measured axially parallel to the central longitudinal axis of the stent 10) and diameters (measured radially from the central longitudinal axis of the stent). For instance, any or all of the proximal flange 30, proximal support flange 31, distal flange 40, distal support flange 41 and any other additional flanges contemplated by the present disclosure may range from 10 mm to 50 mm in diameter and may also range from 0.5 mm to 5 mm in width. Any value within the aforementioned ranges is contemplated as a possible diameter and width for any or all of the proximal flange 30, proximal support flange 31, distal flange 40, distal support flange 41 and any other additional flanges contemplated by the present disclosure.

In some instances, the width of the proximal support flange 31 may be different than (e.g., greater than or less than) the width of the proximal flange 30, and/or the diameter of the proximal support flange 31 may be different than (e.g., greater than or less than) the diameter of the proximal flange 30. In some instances, the width of the distal support flange 41 may be different than (e.g., greater than or less than) the width of the distal flange 40, and/or the diameter of the distal support flange 41 may be different than (e.g., greater than or less than) the diameter of the distal flange 40. For exemplary purposes, FIG. 3 illustrates the proximal flange 30 as having a greater diameter and width than the proximal support flange 31. Such a configuration may also be present with regard to the distal flange 40 and distal support flange 41 (e.g., the distal flange 40 may have a greater diameter and width than the distal support flange 41). For exemplary purposes, FIG. 3 illustrates the distal flange 40 as having a smaller diameter and greater width than the distal support flange 41. Such a configuration may also be present with regard to the proximal flange 30 and proximal support flange 31 (e.g., the proximal flange 30 may have a smaller diameter and greater width than the proximal support flange 31). Other configurations of the flanges are also contemplated, but not illustrated.

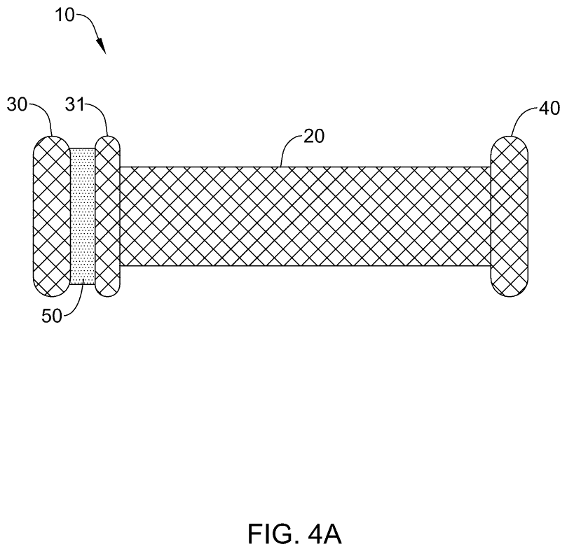

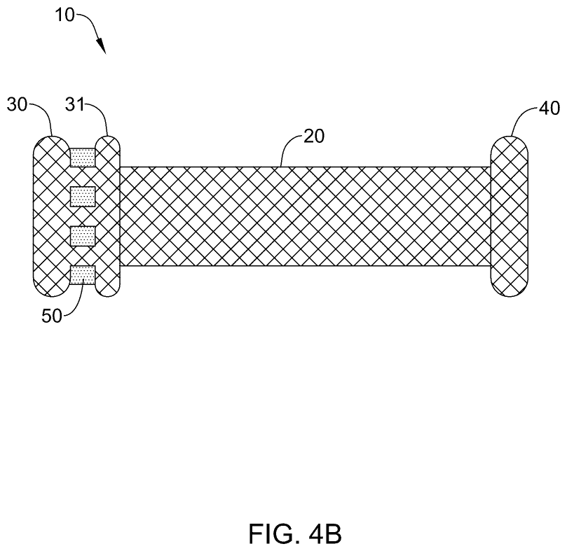

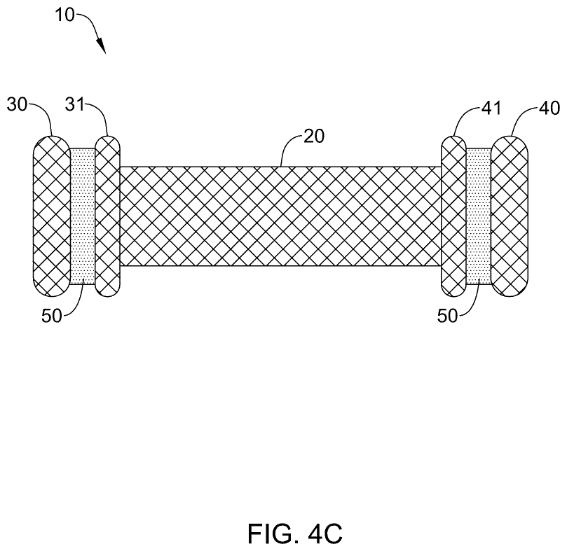

FIGS. 4A-4C show an array of configurations of self-expanding stents according to the present disclosure. Shown in FIG. 4A is a self-expanding stent 10 which includes a saddle region 20, a proximal flange 30, a proximal support flange 31, a distal flange 40, and an open-cell foam material 50 disposed about an exterior of the self-expanding stent 10. The open-cell foam material 50 may be positioned on a desired portion of the exterior of the self-expanding stent 10 configured to be subjected to direct contact with body tissue of the individual in which the stent 10 is implanted. For example, the open-cell foam material 50 may be disposed on or adjacent to the proximal flange 30 and/or disposed on or adjacent to the distal flange 40. In these instances, the open-cell foam material 50 may be arranged on the exterior of the stent 10 so that at least one portion of the open-cell foam material 50 and at least one portion of the proximal flange 30 are visible from the exterior of the self-expanding stent 10. In other instances, the open-cell foam material 50 may be arranged on the exterior of the stent 10 so that at least one portion of the open-cell foam material 50 and at least one portion of the distal flange 40 are visible from the exterior of the self-expanding stent 10. The open-cell foam material 50 may be positioned on the exterior of the stent 10 in a variety of configurations. For example, the open-cell foam material 50 may be interspersed within the proximal flange intermittently, or may be deposited in the form of a ring-shaped or annular construction of open-cell foam material extending entirely around the circumference of the stent 10.

As shown in FIG. 4A, the open-cell foam material 50 (shown as an annular ring of foam material extending entirely around the circumference of the stent 10) may be positioned in the inter-flange space or gap between the proximal flange 30 and the proximal support flange 31. In other embodiments, the open-cell foam material 50 may be positioned in the inter-flange space or gap between the distal flange 40 and the distal support flange 41. In some instances, the open-cell foam material 50 may be recessed within the gap between the proximal flange 30 and the proximal support flange 31 such that the open-cell foam material 50 extends outward a radial distance less than the radial distance of the outermost extent of the proximal flange 30 and/or the proximal support flange 31. In other instances, the open-cell foam material 50 may be positioned in the gap between the proximal flange 30 and the proximal support flange 31 and extend outward a radial distance greater than the radial distance of the outermost extent of the proximal flange 30 and/or the proximal support flange 31. In yet other instances, the open-cell foam material 50 may be positioned in the gap between the proximal flange 30 and the proximal support flange 31 such that the open-cell foam material 50 is flush with the outermost extent of the proximal flange 30 and/or the proximal support flange 31. Although not illustrated in FIG. 4A, a similar configuration may be provided with an open-cell foam material 50 positioned between the distal flange 40 and the distal support flange 41 in some instances.

As shown in FIG. 4B, the open-cell foam material 50 may be intermittently disposed around the circumference of the stent 10 such that a plurality of open-cell foam material segments are circumferentially spaced apart around the circumference of the stent 10. The intermittent segments of open-cell foam material 50 (shown as interspersed segments of foam material) may be positioned in the inter-flange space or gap between the proximal flange 30 and the proximal support flange 31. In other embodiments, the intermittent segments of open-cell foam material 50 may be positioned in the gap between the distal flange 40 and the distal support flange 41. In some instances, the intermittent segments of open-cell foam material 50 may be recessed within the gap between the proximal flange 30 and the proximal support flange 31 such that the intermittent segments of open-cell foam material 50 extend outward a radial distance less than the radial distance of the outermost extent of the proximal flange 30 and/or the proximal support flange 31. In other instances, the intermittent segments of open-cell foam material 50 may be positioned in the gap between the proximal flange 30 and the proximal support flange 31 and extend outward a radial distance greater than the radial distance of the outermost extent of the proximal flange 30 and/or the proximal support flange 31. In yet other instances, the intermittent segments of open-cell foam material 50 may be positioned in the gap between the proximal flange 30 and the proximal support flange 31 such that the intermittent segments of open-cell foam material 50 are flush with the outermost extent of the proximal flange 30 and/or the proximal support flange 31.

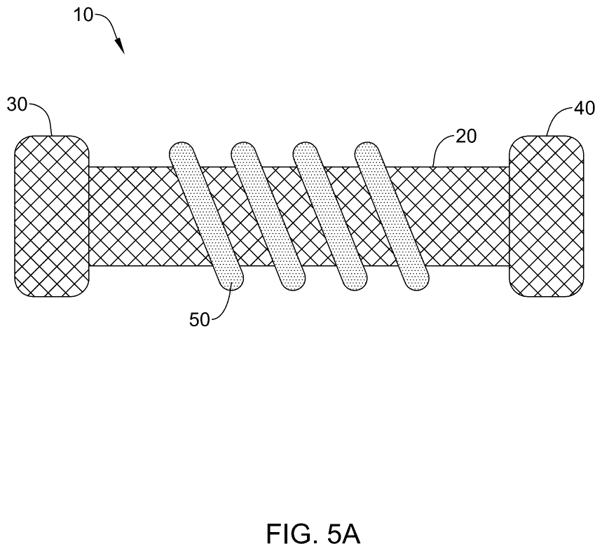

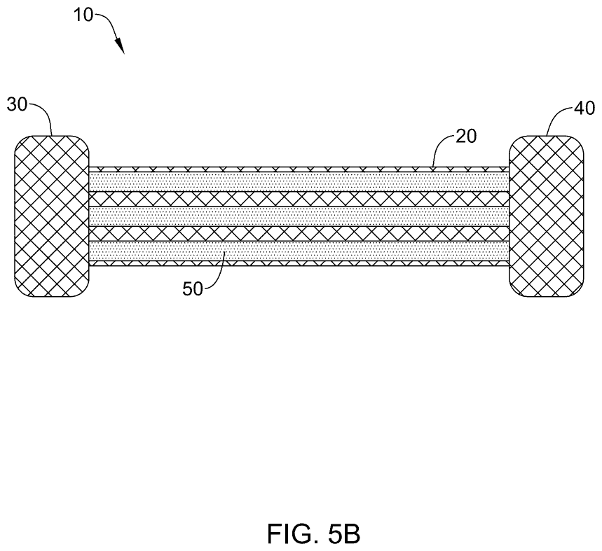

As shown in FIG. 4C, the open-cell foam material 50 (shown as an annular ring of foam material extending entirely around the circumference of the stent 10) may be positioned in the inter-flange space or gap between the proximal flange 30 and the proximal support flange 31. Additionally, the open-cell foam material 50 may be positioned in the inter-flange space or gap between the distal flange 40 and the distal support flange 41. As discussed above, the open-cell foam material 50 may be recessed within the gaps such that the open-cell foam material 50 extends outward a radial distance less than the radial distance of the outermost extent of the flanges. In other instances, the open-cell foam material 50 may be positioned in the gap between flanges such that the open-cell foam material 50 extends outward a radial distance greater than the radial distance of the outermost extent of the flanges. In yet other instances, the open-cell foam material 50 may be positioned in the gap between flanges such that the open-cell foam material 50 is flush with the outermost extent of the flanges. The open-cell foam material 50 shown in FIG. 5C is illustrated as a continuous annular ring extending entirely around the circumference of the stent, however, in other instances, the open-cell foam material 50 may be arranged intermittently around the circumference of the stent 10, as described with respect to FIG. 4B.

In some instances, the stent 10 may be devoid of the proximal support flange 31 and/or the distal support flange 41, in which case the open-cell foam material 50 may be disposed on and contact a surface of the proximal flange 30 and/or the distal flange 40, and be exposed to an exterior of the stent 10. For instance, the open-cell foam material 50 may be disposed on the radially extending wall of the proximal flange 30 facing the distal end (and thus the distal flange 40) of the stent 10 and/or the open-cell foam material 50 may be disposed on the radially extending wall of the distal flange 40 facing the proximal end (and thus the proximal flange 30) of the stent 10.

The open-cell foam material 50 may also be disposed on the stent 10 (e.g., deposed on and in contact with the proximal flange 30, the distal flange 40, and/or the saddle region 20 in a helical pattern, a serpentine pattern, a raised pattern, an embedded pattern, or a pattern in which at least one portion of the open-cell foam material 50 is visible from exterior of the self-expanding stent 10, a pattern in which at least two portions of the open-cell foam material 50 are visible from exterior of the self-expanding stent 10, a pattern in which at least three portions of the open-cell foam material 50 are visible from exterior of the self-expanding stent 10, a pattern in which at least four portions of the open-cell foam material 50 are visible from exterior of the self-expanding stent 10, or a pattern in which at least five or more portions of the open-cell foam material 50 are visible from exterior of the self-expanding stent).

Additionally or alternatively, the open-cell foam material 50 may also be disposed on the stent 10 (e.g., affixed to and in contact with the proximal flange 30, the distal flange 40, and/or the saddle region 20 in a pattern in which no more than one portion of the open-cell foam material 50 is visible from outside the self-expanding stent 10). In other instances, the open-cell foam material 50 may be disposed on the stent 10 (e.g., affixed to and in contact with the proximal flange 30, the distal flange 40, and/or the saddle region 20 in a pattern in which no more than two portions of the open-cell foam material 50 are visible from outside the self-expanding stent 10; in a pattern in which no more than three portions of the open-cell foam material 50 are visible from outside the self-expanding stent 10; in a pattern in which no more than four portions of the open-cell foam material 50 are visible from outside the self-expanding stent; in a pattern in which no more than five portions of the open-cell foam material 50 are visible from outside the self-expanding stent 10).

Additionally or alternatively, the open-cell foam material 50 may be interspersed according to any of the above patterns, and interspersed with any or all of the proximal flange 30, proximal support flange 31, distal flange 40, distal support flange 41, along the saddle region 20 and with any additional other flanges and features contemplated by the present disclosure.

The open-cell foam material 50 of the present disclosure may be applied to any or all embodiments and aspects of the present disclosure. The open-cell foam material 50 may include open cells or voids defined between a cellular matrix in which the open cells or voids are open to the exterior of the open-cell foam material 50 to permit tissue ingrowth into the open cells or voids of the open-cell foam material 50. The open-cell foam material 50 may be any suitable type of open-cell foam, including but not limited to: soft foam, rigid foam, reticular foam, foam core, syntactic foam, expandable foam, coated foam, non-coated foam, an open-cell foam spray, an open-cell foam adhesive, an open-cell foam epoxy, an open-cell foam paint, an open-cell foam application, or any suitable or equivalent open-cell foam or open-cell foam material desired.

As noted above, a space between the proximal flange 30 and the proximal support flange 31 may form a proximal inter-flange space. Open-cell foam material 50 may be disposed within the proximal inter-flange space, between the proximal flange 30 and the proximal support flange 31. Additionally or alternatively, a space between the distal flange 40 and the distal support flange 41 may form a distal inter-flange space. Open-cell foam material 50 may be disposed within the distal inter-flange space, between the distal flange 40 and the distal support flange 41. The open-cell foam material 50 disposed within the proximal inter-flange space and/or the distal inter-flange space may be ring-shaped or annular as it may be in other embodiments, aspects and configurations of self-expanding stent 10. Further, the open-cell foam material of the present disclosure may be employed in any conceivable shape or geometry, including but not limited to: circular, disk-shaped, square, spheroid, obloid, flat, curvilinear, pentagonal, hexagonal, octagonal, elliptical, cubed, cuboid, pyramidal, prismatic or any geometrical construction of the like or equivalent. The open-cell foam material 50 of the present disclosure may also extend from one or more of the proximal flange 30, proximal support flange 31, distal flange 40, and distal support flange 41 in various ways. These ways include, but are not limited to: longitudinally, radially, circumferentially, helically, curvilinear, rectilinear, zig-zagged, undulating, oscillating, sinusoidal, hyperbolically, intermittently, discontinuously, continuously, step-wise, or any other extension patterns desired.

FIGS. 5A and 5B show yet further aspects of the present disclosure. In FIG. 5A, a self-expanding stent 10 is shown with a saddle region 20 extending between a proximal flange 30 and a distal flange 40, and an open-cell foam material 50 affixed to, exposed to, or embedded within the tubular body along at least a portion of the saddle region 20. As shown in FIG. 5A, the open-cell foam material 50 may extend longitudinally from the proximal flange 30 in a distal direction. Additionally or alternatively, the open-cell foam material 50 may also extend longitudinally from the distal flange 40 in a proximal direction. In some instances, the open-cell foam material 50 may be a plurality of longitudinal strips of open-cell foam material extending longitudinally along the saddle region 20 with individual strips circumferentially spaced apart around the circumference of the saddle region 20. In some instances, the longitudinal strips of open-cell foam material 50 may extend along an entire length of the saddle region 20 from the proximal flange 30 to the distal flange 40, or along only a portion of the length between the proximal flange 30 and the distal flange 40.

It is also contemplated that the open-cell foam material 50 may extend longitudinally in a helical pattern (shown in FIG. 5B) along the saddle region 20. For instance, one or more helical strips of open-cell foam material 50 may extend helically along an entire length of the saddle region 20 from the proximal flange 30 to the distal flange 40, or along only a portion of the length between the proximal flange 30 and the distal flange 40. In other instances, the open-cell foam material 50 may extend along the saddle region 20 in other patterns, such as a serpentine pattern between the proximal flange 30 and the distal flange 40.

The open-cell foam material 50 may also longitudinally extend from a proximal support flange 31 and/or a distal support flange 41 (if present), or any number of flanges contemplated by the present disclosure. Further, the open-cell foam material 50 may also extend longitudinally in a helical or serpentine pattern from a proximal support flange 31 and/or a distal support flange 41 (if present), or any number of flanges or features contemplated by the present disclosure. Other patterns of presentation and implementation of the open-cell foam material 50 are also contemplated. Patterns include but are not limited to: intermittent patterns, irregular patterns, checkerboard patterns, oval patterns, circular patterns, striped patterns, dotted patterns, projecting patterns, embedded patterns, raised patterns, disconnected patterns, relief patterns, discontinuous patterns, curvilinear patterns, rectilinear patterns or any pattern desired suitable for the purposes of the present disclosure.

In addition to the flanges listed above, myriad other flange configurations, flange combinations, flange count and flange arrays are contemplated. In certain instances, the self-expanding stent 10 may only include a proximal flange 30. In other instances, the self-expanding stent 10 may include a proximal flange 30 and a distal flange 40. In yet other instances, the self-expanding stent may further include a proximal support flange 31 positioned near the proximal flange 30. In other instances, the self-expanding stent may further include a distal support flange 41 positioned near the distal flange 40. In yet other instances, the self-expanding stent 10 may include all four aforementioned flanges: proximal flange 30, proximal support flange 31, distal flange 40, and distal support flange 41. In yet additional instances, the self-expanding stent 10 may include additional flanges.

Further, any or all flanges contemplated by the present disclosure may be angled with respect to the central longitudinal axis of the tubular body of the self-expanding stent 10. For instance, any or all flanges contemplated by the present disclosure may be angled at an acute angle with respect to the central longitudinal axis of the tubular body of the self-expanding stent, such as by an angle ranging between 15 and 75 degrees, or between 30 and 60 degrees, for example. Other angles for any or all flanges of the present disclosure are also contemplated. The acute angles for any or all flanges contemplated by the present disclosure may be any angle or combination or array of acute angles with respect to the central longitudinal axis of the tubular body of the self-expanding stent 10.

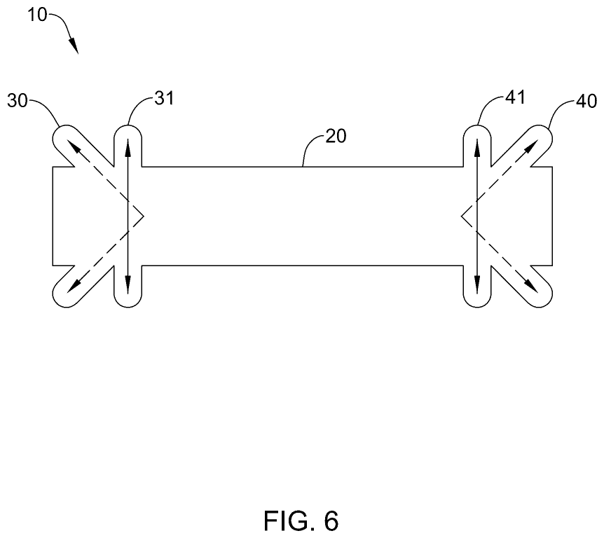

FIG. 6 shows a self-expanding stent 10 including a proximal flange 30 and a distal flange 40, each extending at an acute angle to the central longitudinal axis of the tubular body of the stent 10. FIG. 6 illustrates a self-expanding stent 10 with a saddle region or saddle portion 20 extending between the proximal flange 30 and the distal flange 40, and which further includes a proximal support flange 31 and a distal support flange 41. In this and other instances, the proximal flange 30 and the distal flange 40 may be angled with respect to the central longitudinal axis (labeled X) of the tubular body of the stent 10 at an acute angle. The angle of the proximal flange 30 and the distal flange 40 may range between 30 to 60 degrees with respect to the central longitudinal axis X of the tubular body, for instance. However, other ranges of angles are contemplated. In certain instances, the acute angle of any of the proximal and distal flanges of the self-expanding stent 10 may range from 10 to 70 degrees. In yet other instances, the acute angle of any of the flanges of the self-expanding stent 10 may range from 5 to 85 degrees, for instance.

When describing a flange as extending at an acute angle to the central longitudinal axis X, it is intended to mean that the overall trajectory of the flange lies in a plane that extends at an acute angle such that the radially outer extent of the flange is longitudinally spaced relative to the base of the flange (longitudinally offset from the base of the flange). In some instances, the flange may be angled such that both side-walls of the double-walled flange extend at an acute angle to the central longitudinal axis X.

Angled flanges and flanges extending at an angle in the present disclosure may also be referred to as sloped flanges. In this and other examples, the sloped or angled flanges of the present disclosure may be formed to follow the slope of an imaginary line or predetermined oblique angle projecting from the central longitudinal axis X of the tubular body of the stent 10. The slope of the imaginary line or predetermined angle may be in the proportion of 1 unit in the radial direction to 1 unit in the axial direction, whereby the unit may be a length unit of measurement. The slope proportions may be represented in numerical format such as 1:1, meaning a slope of one radial unit to one axial unit. Other slope proportions contemplated include but are not limited to 2:1, 1:2, 2:3, 3:2, 3:1, 1:3, 1.5:1, 1:1.5, 1.75:1.5, 1.25:1, 1:1.25, 4:1, 5:1, 1:4, 1:5 or any combination or permutation of proportions and/or ratios as desired.

FIG. 6 also illustrates the proximal support flange 31 and the distal support flange 41 extending perpendicular to the central longitudinal axis X. In other words, the proximal support flange 31 may lie in a plane that extends perpendicular to the central longitudinal axis X and/or the distal support flange 41 may lie in a plane that extends perpendicular to the central longitudinal axis X. Thus, in some instances, the stent 10 may include a proximal support flange 31 extending perpendicular to the central longitudinal axis X (e.g., lying in a plane that is perpendicular to the central longitudinal axis X) and a proximal flange 30 extending at an acute angle to the central longitudinal axis X (e.g., lying in a plane that is oblique to the central longitudinal axis X), providing an acute angle between the plane that the proximal support flange 31 lies in and the plane that the proximal flange 30 lies in. Furthermore, the stent 10 may include a distal support flange 41 extending perpendicular to the central longitudinal axis X (e.g., lying in a plane perpendicular to the central longitudinal axis X) and a distal flange 40 extending at an acute angle to the central longitudinal axis X (e.g., lying in a plane that is oblique to the central longitudinal axis X), providing an acute angle between the plane that the distal support flange 41 lies in and the plane that the distal flange 40 lies in.

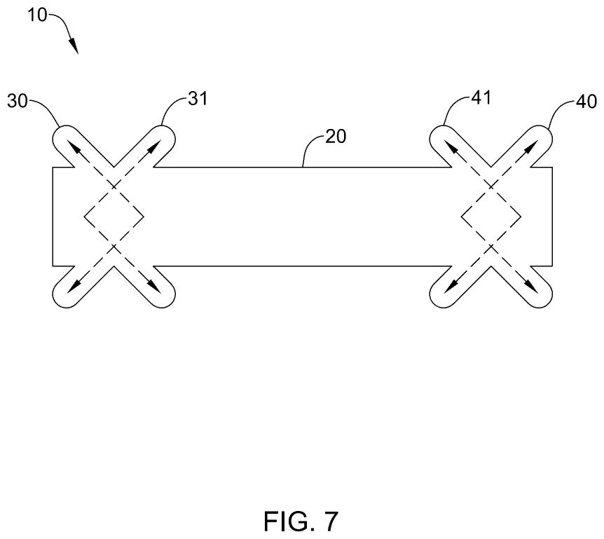

FIG. 7 shows an example of a self-expanding stent 10 in which the flanges (proximal flange 30, proximal support flange 31, distal flange 40 and distal support flange 41) are each angled at an oblique angle to the central longitudinal axis X of the stent 10. The angle of these flanges may range between 15 and 75 degrees, or 30 to 60 degrees with respect to the central longitudinal axis X of the tubular body, for example. However, other ranges of angles are contemplated. In certain instances, the acute angle of any of the flanges of the self-expanding stent 10 may range from 10 to 70 degrees, for example. In yet other instances, the acute angle of any of the flanges of the self-expanding stent 10 may range from 5 to 85 degrees, for example.

As shown in FIG. 7, the proximal flange 30 may be angled toward the proximal end of the stent 10 with the base of the proximal flange 30 located closer to the distal end of the stent 10 than the outermost extent of the proximal flange 30. Likewise, the distal flange 40 may be angled toward the distal end of the stent 10 with the base of the distal flange 40 located closer to the proximal end of the stent 10 than the outermost extent of the distal flange 40. The proximal support flange 31 may be angled distally from the base of the proximal flange 30 such that the proximal support flange 31 is angled toward the distal end of the stent 10 with the base of the proximal support flange 31 located closer to the proximal end of the stent 10 than the outermost extent of the proximal support flange 31. Likewise, the distal support flange 41 may be angled proximally from the base of the distal flange 40 such that the distal support flange 41 is angled toward the proximal end of the stent 10 with the base of the distal support flange 41 located closer to the proximal end of the stent 10 than the outermost extent of the distal support flange 41. Thus the proximal flange 30 may extend at an acute angle to the central longitudinal axis X (e.g., lie in a plane that is oblique to the central longitudinal axis X), the distal flange 40 may extend at an acute angle to the central longitudinal axis (e.g., lie in a plane that is oblique to the central longitudinal axis X), the proximal support flange 31 may extend at an acute angle to the central longitudinal axis X (e.g., lie in a plane that is oblique to the central longitudinal axis X), and/or the distal support flange 41 may extend at an acute angle to the central longitudinal axis X (e.g., lie in a plane that is oblique to the central longitudinal axis X).

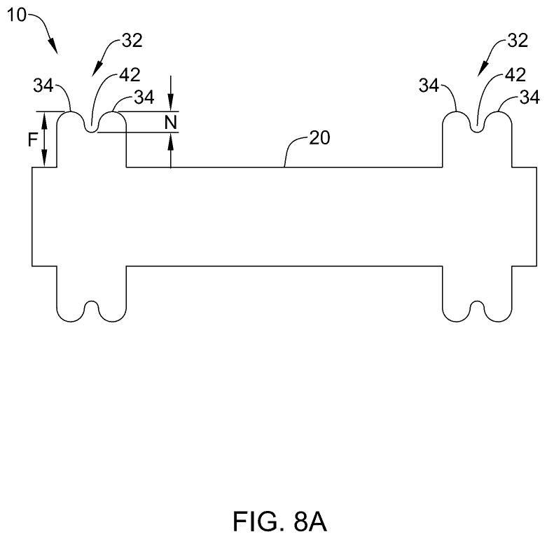

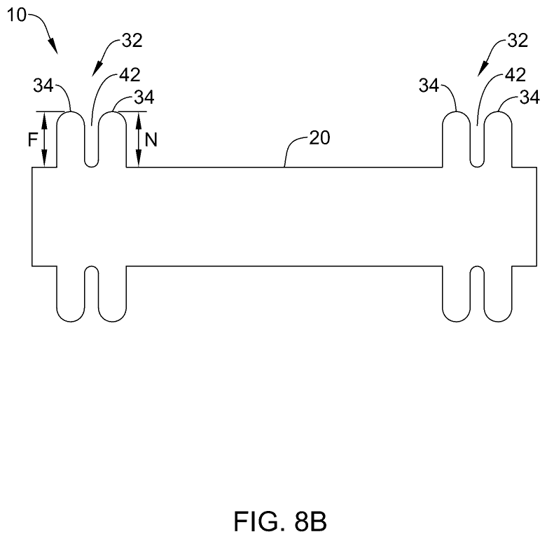

FIG. 8A shows an example of a self-expanding stent 10 in which the flanges 32 are grooved and/or formed as a grooved flange construction, including a circumferential groove 42 arranged between a first flange portion 34 (e.g., a first toroidal flange portion) and a second flange portion 34 (e.g., a second toroidal flange portion). Thus, the circumferential groove 42 may axially space apart a first flange portion 34 (e.g., a first toroidal flange portion) and a second flange portion 34 (e.g., a second toroidal flange portion) of the flange 32. In other words, the flanges 32 of this and other examples may incorporate one or more grooves 42 or concave channel structures (e.g., circumferential grooves or channels extending around the circumference of the flange 32) which may provide stent retention and/or impede stent migration without causing undue trauma or damage to patient tissue (i.e., lumen, organ or blood vessel walls) contacted by the flanges 32. The flanges 32 of this and other examples may have one or more concave depressions (e.g., circumferential grooves 42) which allow for tissue ingrowth within the one or more concave depressions (e.g., circumferential grooves 42) which may provide stent retention and/or impede stent migration without causing undue trauma or damage to patient tissue. Equivalent to a groove, any channel, convexity, concavity, undulating depression or recess may be employed within any or all of the flanges contemplated by the present disclosure and further may be employed in a plurality of flanges and may be provided in any combination or permutation of one or more grooves, channels, concavities, undulating depressions or recesses.

The groove 42 (e.g., channel, concavity, undulating depression or recess) of any and/or all flanges 32 (including other flanges contemplated by the present disclosure) may be designed with a minimum and/or maximum depth N. It can be appreciated that the minimum and/or maximum depth N is calculated as the distance between the apex (i.e., radially outermost extent) of the flange 32 (e.g., the outermost extent of the first and/or second flange portion 34 of the flange 32) to the nadir or base (i.e. radially inwardmost extent) of the groove 42. The depth N of the groove 42 may be less than the radial height F of the flange 32 (e.g., the radial height of the first and/or second flange portion 34 of the flange 32). For instance, the depth N of the groove 42 may be 75% or less, 60% or less, 50% or less, or 30% or less than the radial height F of the flange 32. In some instances, the depth N of the groove 42 may be 1 mm or less, 2 mm or less, 3 mm or less, 4 mm or less, or 5 mm or less, for example. In this and other examples, the depth N of the groove 42 may be of a depth such that the nadir or base of the groove 42 is radially outward of the outer diameter of the saddle region 20 of the tubular body of stent 10.

Any or all flanges contemplated by the present disclosure may assume similar or identical construction to those disclosed by FIG. 8A. In other words, and not by limitation, the flanges 32 of FIG. 8A may further be angled or oriented to any contemplated angle or orientation contemplated by the present disclosure. The flanges 32 of FIG. 8A may further be present in any amount contemplated by the present disclosure. Alternatively or additionally, the flanges 32 of FIG. 8A may further include an open-cell foam material 50 disposed within or disposed on any element and on or within any region or totality of the flanges 32. Open-cell foam material 50 may further extend from any or all flanges 32 and may extend in any direction from any or all flanges 32. For example, open-cell foam material 50 may extend from any or all flanges 32 in a longitudinal direction, in a radial direction, in a circumferential direction, in a lateral direction, in a serpentine pattern, in a helical pattern, or any extension pattern or direction as desired.

FIG. 8B shows an example of a self-expanding stent 10 in which the flanges 32 are divided or are formed in a split-flange construction including a circumferential groove 42 arranged between a first flange portion 34 (e.g., a first toroidal flange portion) and a second flange portion 34 (e.g., a second toroidal flange portion). Thus, the circumferential groove 42 may axially space apart a first flange portion 34 (e.g., a first toroidal flange portion) and a second flange portion 34 (e.g., a second toroidal flange portion) of the flange 32. As shown in FIG. 8B, the groove 42 (e.g., channel, concavity, undulating depression or recess) of any and/or all flanges 32 (including other flanges contemplated by the present disclosure) may be designed with a minimum and/or maximum depth N. It can be appreciated that the minimum and/or maximum depth N is calculated as the distance between the apex (i.e., radially outermost extent) of the flange 32 (e.g., the outermost extent of the first and/or second flange portion 34 of the flange 32) to the nadir or base (i.e. radially inwardmost extent) of the groove 42. The depth N of the groove 42 may be equal to or substantially equal to the radial height F of the flange 32 (e.g., the radial height of the first and/or second flange portion 34 of the flange 32). For instance, the depth N of the groove 42 may be within 5% or less, within 2% or less, or within 1% or less than the radial height F of the flange 32. In some instances, the depth N of the groove 42 may be 10 mm or less, 8 mm or less, or 5 mm or less, for example. In this and other examples, the depth N of the groove 42 may be of a depth such that the nadir or base of the groove 42 is substantially equal to the outer diameter of the saddle region 20 of the tubular body of stent 10.

In some instances, the groove 42 in the proximal flange 32 may extend to a different depth N than the groove 42 of the distal flange 32. For instance, a proximal flange 32 may include a groove 42 extending to a depth N greater than or less than a depth N of a groove 42 of a distal flange 32. In yet further non-limiting examples, the depth N of the groove(s) 42 may be greater than the radial height F of the flange 32 such that the groove(s) projects radially inward of the outer diameter of the saddle region 20 and into the tubular body of stent 10.

Any or all flanges contemplated by the present disclosure may assume similar or identical construction to those disclosed by FIG. 8B. In other words, and not by limitation, it can be appreciated that the flanges 32 of FIG. 8B may further be angled or oriented to any contemplated angle or orientation contemplated by the present disclosure. The flanges 32 of FIG. 8B may further be present in any amount contemplated by the present disclosure. Alternatively or additionally, the flanges 32 of FIG. 8B may further include an open-cell foam material 50 disposed within or disposed on any element and on or within any region or totality of the flanges 32. Open-cell foam material 50 may further extend from any or all flanges 32 and may extend in any direction from any or all flanges 32. For example, open-cell foam material 50 may extend from any or all flanges 32 in a longitudinal direction, in a radial direction, in a circumferential direction, in a lateral direction, in a serpentine pattern, in a helical pattern, or any extension pattern or direction desired.

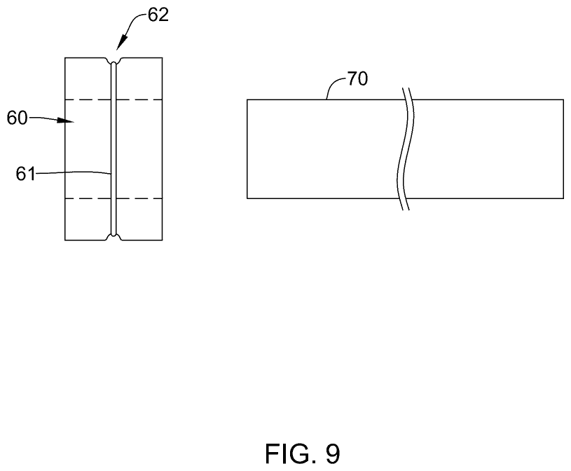

FIG. 9 illustrates an example device and method for forming (e.g., heat setting) an exemplary stent 10 of the present disclosure through an annealing process incorporating a stent scaffold or stenting material. FIG. 9 shows a device and method that further includes at least a flange ring 60, a central mandrel 70 and a retention wire 61. Retention wire 61 may be positioned or positionable within an annular groove 62 of flange ring 60, The flange ring 60 may be positioned along central mandrel 70. The interwoven wires forming the expandable framework (e.g., stent scaffold) of the stent 10 may be disposed on the mandrel 70 and the flange ring 60 such that the expandable framework surrounds the mandrel 70 and the flange ring 60. The portion of the expandable framework intended to form a proximal or distal flange of the stent 10 may surround the flange ring 60. The central mandrel 70 may possess an outer diameter that is equal to or substantially equal to the desired diameter of the saddle region 20 of stent 10. The retention wire 61 may then be circumferentially disposed around the expandable framework with the retention wire 61 aligned with the groove 62. The retention wire 61 may be tightened or otherwise reduced in diameter to draw portions of the interwoven wires of the expandable framework into the groove 62, and thus deflect the interwoven wires of the flange region radially inward toward the central longitudinal axis of the stent 10 and mandrel 70. The expandable framework may be held in the groove 62 with the retention wire 61 while subjecting the expandable framework to an annealing process in which the stent material is exposed to extreme heat to heat set the expandable framework to form a flange with a groove as described above.

In other words, FIG. 9 illustrates an example stent shaping central mandrel 70 which may further operatively include a flange ring 60 configured for removable attachment to the stent shaping central mandrel 70. The flange ring 60 may further incorporate a retention wire 61 which holds a portion of the expandable framework of the stent 10 the groove 62 of the flange ring 60 during the annealing process so that the combination of flange ring 60 and retention wire 61 form a grooved flange of the stent 10 of the present disclosure by heat setting the material comprising stent 10.

As will be illustrated and described below, stent shaping central mandrel 70 may be utilized to change the shape (e.g., form) of a straight, cylindrical tubular, braided, knitted or woven stent scaffold into a more complex-shaped stent 10 as disclosed herein. In order to form the shape of the stent (for instance as illustrated in FIG. 1), it may be desirable to utilize an annealing process (i.e., exposing the stent 10 to extreme heat) to manipulate the various components of the stent scaffold to form stent 10 of the present disclosure.

An example method to form stent 10 of the present disclosure may include positioning a straight, cylindrical tubular braided, knitted, woven or interwoven stent scaffold around the stent shaping central mandrel 70, including the flange ring 60, followed by the application of a heat treatment to anneal the stent filaments of the stent scaffold in a preferred shape (e.g., the shape dictated by the stent shaping central mandrel 70 and the flange ring 60 (and groove 62, if present). The stent scaffold may be formed from one or more interwoven filaments formed into a cylindrical structure. The stent scaffold is then placed along the stent shaping central mandrel 70 and surrounds the flange ring 60. The retention wire 61 is then disposed circumferentially around the stent scaffold radially outward of the groove 62. Upon tightening the retention wire 61 or otherwise manipulating the retention wire, the wires of the stent scaffold are deflected radially inward into the groove 62. Thet stent scaffold may then be subjected to an annealing process by applying heat to heat set (i.e., anneal) the stent scaffold into stent 10 and to anneal one or more sections of the stent scaffold into one or more flanges formed over the flange ring 60 that defect into the groove 62. Once the stent scaffold is fully heat set and formed into stent 10, stent 10 is removed from the mandrel and ready for use and deployment.

It can be appreciated from FIG. 9 that the annealing process disclosed herein can be implemented for any and all flange and stent designs and constructions contemplated by the present disclosure. The process outlined in FIG. 9 and the accompanying disclosure may be utilized to form additional flanges than those illustrated in the drawings. The process outlined in FIG. 9 and the accompanying disclosure may also be utilized to form angled flanges, a series of flanges of differing angles, or any combination or permutation of flanges bearing identical or varying angles.

Any or all flanges contemplated by the present disclosure may be present in a variety of diameters and thicknesses. Further, the outer extent or tissue-contacting surfaces of any or all flanges contemplated by the present disclosure may be angled, beveled, rounded, inflated, widened, narrowed, concave, convex, slit open, split apart, wedged, smoothened, roughened, lubricious, coated, or have its surface modified by any known manner in the art.

In some embodiments, in the deployed configuration, the proximal flange 30 (and/or the proximal support flange 31, distal flange 40 and distal support flange 41) may be configured to resist collapsing radially inward under a first radial inward force, the distal flange 40 (and/or proximal support flange 31 and distal support flange 41) may be configured to resist collapsing radially inward under a second radial inward force, and the saddle portion 20 may be configured to resist collapsing radially inward under a third radial inward force less than the first radial inward force and the second radial inward force. In some embodiments, the first radial inward force may be within about 25% of the second radial inward force. In some embodiments, the first radial inward force may be within about 10% of the second radial inward force. In some embodiments, the first radial inward force may be within about 5% of the second radial inward force. In some embodiments, the third radial inward force may be less than about 75% of the first radial inward force and/or the second radial inward force. In some embodiments, the third radial inward force may be less than about 50% of the first radial inward force and/or the second radial inward force. In some embodiments, the first radial inward force and/or the second radial inward force may be about 300% to about 500% greater than the third radial inward force. Other configurations are also contemplated.

In some embodiments, the tubular body of the self-expanding stent 10 may define an outer surface that may extend, sequentially, from the proximal end: longitudinally, radially outward, curve back on itself to radially inward, longitudinally toward the distal end, radially outward, curve back on itself to radially inward, and longitudinally to the distal end. In other words, each flange (i.e., proximal flange 30 and distal flange 40) may include first and second radially extending wall portions longitudinally spaced apart from one another with an apical curved region spanning therebetween at the outer extent or tissue-contacting surface of the flange, with a first radially extending wall portion extending radially outward from the central longitudinal axis X to an outer extent of the flange and the second radially extending wall portion extending radially inward from the outer extent of the flange toward the central longitudinal axis X. Other configurations are also contemplated.

In some embodiments, the tubular body of the self-expanding stent 10 may define an outer surface that may extend, sequentially, from the first end: longitudinally, radially outward, curve back on itself to radially inward, longitudinally, radially outward, curve back on itself to radially inward, longitudinally, radially outward, curve back on itself to radially inward, longitudinally, radially outward, curve back on itself to radially inward, and longitudinally, to the second end. In other words, each flange (i.e., proximal flange 30, proximal support flange 31, distal flange 40 and distal support flange 41) may include first and second radially extending wall portions longitudinally spaced apart from one another with an apical curved region spanning therebetween at the outer extent or tissue-contacting surface of the flange, with a first radially extending wall portion extending radially outward from the central longitudinal axis X to an outer extent of the flange and the second radially extending wall portion extending radially inward from the outer extent of the flange toward the central longitudinal axis X. Other configurations are also contemplated.

In some embodiments, the tubular body of the self-expanding stent 10 may be formed from a plurality of filaments or wires that may be woven, braided, wound, knitted, meshed, and combinations thereof, around a central longitudinal axis X to form the tubular body. The tubular body of the self-expanding stent 10 may include multiple filaments or wires of a metal material, such as nitinol or nitinol-containing material, or other nickel-titanium alloy, for example. In some instances, the filaments or wires may have a diameter of about 0.01 inches to about 0.025 inches, for example. The number and the diameters of the filaments or wires, which may be the same or different, are not limiting, and other numbers and other diameters of filaments or wires may suitably be used. Desirably, an even number of filaments or wires may be used, for example, from about 2 to about 50 filaments or wires, about 6 to about 40 filaments or wires, about 10 to about 36 filaments or wires, etc.

Desirably, the filaments or wires are made from any suitable implantable biocompatible material, including without limitation nitinol, stainless steel, cobalt-based alloy such as Elgiloy®, platinum, gold, titanium, tantalum, niobium, polymeric materials and combinations thereof. Useful and nonlimiting examples of polymeric stent materials include poly(L-lactide) (PLLA), poly(D, L-lactide) (PLA), poly(glycolide) (PGA), poly(L-lactide-co-D, L-lactide) (PLLA/PLA), poly(L-lactide-co-glycolide) (PLLA/PGA), poly(D, L-lactide-co-glycolide) (PLA/PGA), poly(glycolide-co-trimethylene carbonate) (PGA/PTMC), polydioxanone (PDS), Polycaprolactone (PCL), polyhydroxybutyrate (PHBT), poly(phosphazene) poly(D, L-lactide-co-caprolactone) PLA/PCL), poly(glycolide-co-caprolactone) (PGA/PCL), poly(phosphate ester) and the like. Filaments or wires made from polymeric materials may also include radiopaque materials, such as metallic-based powders, particulates or pastes which may be incorporated into the polymeric material. For example, the radiopaque material may be blended with the polymer composition from which the polymeric filaments or wires are formed, and subsequently fashioned into the tubular body as described herein. Alternatively, the radiopaque material may be applied to the surface of the metal or polymer filaments or wires of the tubular body of the self-expanding stent 10. In either embodiment, various radiopaque materials and their salts and derivatives may be used including, without limitation, bismuth, barium and its salts such as barium sulphate, tantalum, tungsten, gold, platinum and titanium, to name a few. Additional useful radiopaque materials may be found in U.S. Pat. No. 6,626,936, the contents of which are incorporated herein by reference. Metallic complexes useful as radiopaque materials are also contemplated. The tubular body of the self-expanding stent 10 may be selectively made radiopaque at desired areas along the filaments or wires or may be fully radiopaque.

In some instances, the filaments or wires may have a composite construction having an inner core of tantalum, gold, platinum, tungsten, iridium, or combination thereof and an outer member or layer of nitinol to provide a composite wire for improved radiopacity or visibility. In one example, the inner core may be platinum and the outer layer may be nitinol. The inner core of platinum may represent about at least 10% of the filaments or wires based on overall cross-sectional percentage. Moreover, nitinol that has not been treated for shape memory such as by heating, shaping and cooling the nitinol at its martensitic and austenitic phases, is also useful as the outer layer. Further details of such composite wires may be found in U.S. Pat. No. 7,101,392, the contents of which is incorporated herein by reference. The filaments or wires may be made from nitinol, or composite filaments or wires having a central core of platinum and an outer layer of nitinol. Further, the filling weld material, if required by welding processes such as MIG, may also be made from nitinol, stainless steel, cobalt-based alloy such as Elgiloy, platinum, gold, titanium, tantalum, niobium, and combinations thereof. Additional and/or other materials suitable for use in the tubular body of the self-expanding stent are described below.

In some embodiments, the tubular body of the self-expanding stent 10 may optionally include a polymeric cover disposed on at least a portion of the tubular body, the saddle region 20 and/or the expandable framework. In some embodiments, the polymeric cover may be disposed on the saddle portion 20. In some embodiments, the polymeric cover may additionally or alternatively be disposed on the proximal flange 30, the distal flange 40, the proximal support flange 31 and the distal support flange 41. In some embodiments, the polymeric cover may be disposed on the proximal flange 30, the distal flange 40, and the saddle portion 20. In some embodiments, the polymeric cover may be disposed on and/or along an outer surface of the tubular body of the self-expanding stent 10 and/or the expandable framework. In other embodiments, the polymeric cover may be selectively disposed on and/or along an outer surface of the tubular body of the self-expanding stent 10. The selectively disposed polymeric cover may be disposed on and/or along an outer surface of the tubular body of the self-expanding stent 10 in a pattern, an array, intermittently, or on just one element of the self-expanding stent 10, on a pair of elements, on at least three elements, or disposed on at least four elements or more.

In certain instances, the framework of the tubular body of the self-expanding stent 10 may be embedded in the polymeric cover. In some embodiments, the polymeric cover may be disposed on and/or along an inner surface of the tubular body of the self-expanding stent 10 and/or the expandable framework. In some embodiments, the polymeric cover may be fixedly or releasably secured to, bonded to, or otherwise attached to the tubular body of the self-expanding stent 10 and/or the expandable framework. In some embodiments, the polymeric cover may be impermeable to fluids, debris, medical instruments, etc. Some suitable but non-limiting materials for the polymeric cover are described below.

Methods of deploying and implanting a self-expanding stent are also contemplated. In one instance, a method of deploying a self-expanding stent 10 within a body lumen of a patient includes an implanting step. The implanting step further includes implanting a self-expanding stent 10 within a body lumen of a patient by the following process: 1.) radially expanding a proximal flange 30 of the self-expanding stent 10 against a wall of the body lumen. 2.) radially expanding a distal flange 40 of the self-expanding stent 10 against a wall of the body lumen, such that a saddle region 20 of the self-expanding stent 10 extends between the proximal flange 30 and the distal flange 40 of the self-expanding stent 10. Further, the self-expanding stent may also include an open-foam cell material 50 positioned on the self-expanding stent 10 and the open-cell foam material 50 may be adjacent to at least one of the proximal flange 30 and the distal flange 40 to allow tissue ingrowth within the open-cell foam material 50. Allowing tissue ingrowth within the open-cell foam material further aides in resisting stent migration post deployment and implantation of the stent 10 in a body lumen or across body structures.

The open-cell foam material 50 may also be disposed anywhere on or within the self-expanding stent 10 in the methods contemplated by the present disclosure. The open-cell foam material 50 may be embedded or exposed to any element or feature of the self-expanding stent 10 in the methods contemplated by the present disclosure. Further, any positioning or implementation of open-cell foam material described by the present disclosure or known in the art is also contemplated by the methods of the present disclosure.

Alternative or additional methods are also contemplated. In another instance, a method of deploying a self-expanding stent 10 within a body lumen of a patient includes an implanting step. The implanting step further includes implanting a self-expanding stent 10 within a body lumen of a patient by the following process: 1.) radially expanding a proximal flange 30 and a proximal support flange 31 of the self-expanding stent 10 against a wall of the body lumen. 2.) radially expanding a distal flange 40 and a distal support flange 41 of the self-expanding stent 10 against a wall of the body lumen, such that a saddle region 20 of the self-expanding stent 10 extends between the proximal support flange 31 and the distal support flange 41 of the self-expanding stent 10. Further, the self-expanding stent also includes an open-foam cell material 50 positioned on the self-expanding stent 10 and the open-cell foam material 50 may be adjacent to at least one of the proximal flange 30, the proximal support flange 31, the distal flange 40 and the distal support flange 41 to allow tissue ingrowth within the open-cell foam material 50.

Further methods contemplated by the present disclosure include but are not limited to the following example: In yet another instance, a method of deploying a self-expanding stent 10 within a body lumen of a patient includes an implanting step. The implanting step further includes implanting a self-expanding stent 10 within a body lumen of a patient by the following process: 1.) radially expanding a proximal flange 30 and an angled proximal support flange 31 of the self-expanding stent 10 against a wall of the body lumen. The angled proximal support flange 31 residing at an angle between 30 and 60 degrees with respect to the longitudinal axis of the self-expanding stent 10. 2.) radially expanding a distal flange 40 and an angled distal support flange 41 of the self-expanding stent 10 against a wall of the body lumen, such that a saddle region 20 of the self-expanding stent 10 extends between the proximal support flange 31 and the angled distal support flange 41 of the self-expanding stent 10. The angled distal support flange 41 residing at an angle between 30 and 60 degrees with respect to the longitudinal axis of the self-expanding stent 10. Further, the self-expanding stent also includes an open-foam cell material 50 positioned on the self-expanding stent 10 and the open-cell foam material 50 may be adjacent to at least one of the proximal flange 30, the angled proximal support flange 31, the distal flange 40 and the angled distal support flange 41 to allow tissue ingrowth within the open-cell foam material 50.

Another method contemplated by the present disclosure includes but is not limited to the following example: In another aspect, a method of deploying a self-expanding stent 10 within a body lumen of a patient includes an implanting step. The implanting step further includes implanting a self-expanding stent 10 within a body lumen of a patient by the following process: 1.) radially expanding an angled proximal flange 30 and an angled proximal support flange 31 of the self-expanding stent 10 against a wall of the body lumen. The angled proximal flange 30 and the angled proximal support flange 31 residing at an angle between 30 and 60 degrees with respect to the longitudinal axis of the self-expanding stent 10. 2.) radially expanding an angled distal flange 40 and an angled distal support flange 41 of the self-expanding stent 10 against a wall of the body lumen, such that a saddle region 20 of the self-expanding stent 10 extends between the angled proximal support flange 31 and the angled distal flange 41 of the self-expanding stent 10. The angled distal support flange 41 and the angled distal flange 40 residing at an angle between 30 and 60 degrees with respect to the longitudinal axis of the self-expanding stent 10. Further, the self-expanding stent also includes an open-foam cell material 50 positioned on the self-expanding stent 10 and the open-cell foam material 50 may be adjacent to at least one of the angled proximal flange 30, the angled proximal support flange 31, the angled distal flange 40 and the angled distal support flange 41 to allow tissue ingrowth within the open-cell foam material 50.

Additional methods are contemplated by the present disclosure and without limiting the disclosure includes any permutation or combination of flanges, flange angles, degrees of radial flange expansion, flange diameter, flange thickness, self-expanding stent length, self-expanding stent diameter and self-expanding stent thickness. The methods above may also be contemplated for positioning of a self-expanding stent in other suitable areas of a patient, including but not limited to: vessels, organs, ducts, or any anatomical structure suitable for the purposes of the present disclosure. Further, any recited element or feature of the methods disclosed herein are also applicable to the devices (i.e., self-expanding stents, stents, endoprostheses) disclosed herein.

The materials that can be used for the various components of the self-expanding stent 10 and the various elements thereof disclosed herein may include those commonly associated with medical devices. However, this is not intended to limit the devices and methods described herein, as the discussion may be applied to other elements, members, components, or devices disclosed herein, such as, but not limited to, the expandable framework, the first flange portion, the second flange portion, the saddle portion, the polymeric cover, and/or elements or components thereof.

In some embodiments, the self-expanding stent 10, and/or components thereof, may be made from a metal, metal alloy, polymer (some examples of which are disclosed below), a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12(such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyurethane silicone copolymers (for example, ElastEon® from Aortech Biomaterials or ChronoSil® from AdvanSource Biomaterials), biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In some embodiments, a linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Other suitable materials may include ULTANIUM™ (available from Neo-Metrics) and GUM METAL™ (available from Toyota). In some other embodiments, a superelastic alloy, for example a superelastic nitinol can be used to achieve desired properties.

In at least some embodiments, portions or all of the self-expanding stent 10, and/or components thereof, may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the self-expanding stent 10 in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the self-expanding stent 10 to achieve the same result.

In some embodiments, a degree of Magnetic Resonance Imaging (MRI) compatibility is imparted into the self-expanding stent 10 and/or other elements disclosed herein. For example, the self-expanding stent 10, and/or components or portions thereof, may be made of a material that does not substantially distort the image and create substantial artifacts (i.e., gaps in the image). Certain ferromagnetic materials, for example, may not be suitable because they may create artifacts in an MRI image. The self-expanding stent 10, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

In some embodiments, the self-expanding stent and/or other elements disclosed herein may include a fabric material disposed over or within the structure. The fabric material may be composed of a biocompatible material, such a polymeric material or biomaterial, adapted to promote tissue ingrowth. In some embodiments, the fabric material may include a bioabsorbable material. Some examples of suitable fabric materials include, but are not limited to, polyethylene glycol (PEG), nylon, polytetrafluoroethylene (PTFE, ePTFE), a polyolefinic material such as a polyethylene, a polypropylene, polyester, polyurethane, and/or blends or combinations thereof.

In some embodiments, the self-expanding stent 10 and/or other elements disclosed herein may include and/or be formed from a textile material. Some examples of suitable textile materials may include synthetic yarns that may be flat, shaped, twisted, textured, pre-shrunk or un-shrunk. Synthetic biocompatible yarns suitable for use in the present invention include, but are not limited to, polyesters, including polyethylene terephthalate (PET) polyesters, polypropylenes, polyethylenes, polyurethanes, polyolefins, polyvinyls, polymethylacetates, polyamides, naphthalene dicarboxylene derivatives, natural silk, and polytetrafluoroethylenes. Moreover, at least one of the synthetic yarns may be a metallic yarn or a glass or ceramic yarn or fiber. Useful metallic yarns include those yarns made from or containing stainless steel, platinum, gold, titanium, tantalum or a Ni—Co—Cr-based alloy. The yarns may further include carbon, glass or ceramic fibers. Desirably, the yarns are made from thermoplastic materials including, but not limited to, polyesters, polypropylenes, polyethylenes, polyurethanes, polynaphthalenes, polytetrafluoroethylenes, and the like. The yarns may be of the multifilament, monofilament, or spun types. The type and design of the yarn chosen may be selected in a manner which forms a biocompatible and implantable prosthesis and, more particularly, a vascular structure having desirable properties.

In some embodiments, the self-expanding stent 10 and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethylketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl keton, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps without exceeding the scope of the invention. This may include, to the extent that it is appropriate, the use of any of the features of one example embodiment being used in other embodiments. The invention's scope is, of course, defined in the language in which the appended claims are expressed.

What is claimed is:

1. A self-expanding stent, comprising:
a tubular body having a proximal end and a distal end, the tubular body formed of one or more interwoven filaments;
wherein the tubular body comprises a proximal flange near the tubular body proximal end;
wherein the tubular body further comprises a saddle region extending longitudinally and distally from the proximal flange;
wherein the proximal flange further comprises an open-cell foam material affixed within or adjacent to the proximal flange; the open-cell foam material configured to allow tissue ingrowth within the open-cell foam material, thereby further counteracting migration of the self-expanding stent.

2. The self-expanding stent of claim 1, wherein the tubular body further comprises a distal flange near the tubular body distal end.

3. The self-expanding stent of claim 2, wherein the tubular body further comprises a distal support flange situated proximally to and near the distal flange.

4. The self-expanding stent of claim 3, wherein the tubular body further comprises a proximal support flange situated distally to and near the proximal flange.

5. The self-expanding stent of claim 4, wherein a space between the proximal flange and the proximal support flange forms a proximal inter-flange space; and further wherein the open-cell foam material is disposed within the proximal inter-flange space.

6. The self-expanding stent of claim 1, wherein the open-cell foam material extends helically or longitudinally from the proximal flange.

7. The self-expanding stent of claim 1, wherein the open-cell foam material is interspersed within the proximal flange, such that at least one or more portions of the proximal flange and at least one or more portions of open-cell foam material is visible from outside the self-expanding stent.

8. The self-expanding stent of claim 1, wherein the open-cell foam material is ring-shaped or annular.

9. The self-expanding stent of claim 4, wherein at least one or more flanges of the tubular body project from the tubular body at an angle ranging between 30 to 60 degrees with respect to a longitudinal axis of the tubular body.

10. The self-expanding stent of claim 4, wherein at least two or more flanges of the tubular body project from the tubular body at an angle ranging between 30 to 60 degrees with respect to a longitudinal axis of the tubular body.

11. A self-expanding stent, comprising:
a tubular body having a proximal end and a distal end, the tubular body formed of one or more interwoven filaments;
wherein the tubular body comprises a proximal flange near the proximal end and a distal flange near the distal end;
wherein one or more of the proximal flange and the distal flange project from the tubular body at an angle ranging between 30 to 60 degrees;
wherein the tubular body further comprises a saddle region extending between the proximal flange and the distal flange.

12. The self-expanding stent of claim 11, wherein the tubular body further comprises a proximal support flange situated distally to and near the proximal flange; and wherein the tubular body further comprises a distal support flange situated proximally to and near the distal flange.

13. The self-expanding stent of claim 12, wherein at least one or more of the proximal support flange and the distal support flange of the tubular body project from the tubular body at an angle ranging between 30 to 60 degrees with respect to the longitudinal axis of the tubular body.

14. The self-expanding stent of claim 12, wherein both the proximal support flange and the distal support flange of the tubular body project from the tubular body at an angle ranging between 30 to 60 degrees with respect to the longitudinal axis of the tubular body.

15. The self-expanding stent of claim 11, wherein the tubular body further comprises an open-cell foam material affixed to and exposed to an exterior of the tubular body.

16. The self-expanding stent of claim 11, wherein an open-cell foam material is interspersed within the proximal flange, such that at least one or more portions of the proximal flange and at least one or more portions of open-cell foam material is visible from outside the self-expanding stent.

17. The self-expanding stent of claim 12, wherein a space between the proximal flange and the proximal support flange forms a proximal inter-flange space; and further wherein an open-cell foam material is disposed within the proximal inter-flange space.

18. The self-expanding stent of claim 15, wherein the open-cell foam material affixed to and exposed to the exterior of the tubular body extends helically or longitudinally from the proximal flange or proximal support flange.

19. A method of deploying a self-expanding stent within a body lumen of a patient, the method comprising:
implanting a self-expanding stent within a body lumen of a patient,
wherein the implanting step further comprises:
a. radially expanding a proximal flange of the self-expanding stent against a wall of the body lumen;
b. radially expanding a distal flange of the self-expanding stent against a wall of the body lumen, such that a saddle region of the self-expanding stent extends between the proximal flange and the distal flange of the self-expanding stent;
c. wherein an open-cell foam material is positioned on the stent adjacent to at least one of the proximal flange and the distal flange for tissue ingrowth therein.

20. The method of claim 19, wherein the proximal flange has a first diameter and the distal flange has a second diameter; the first diameter being different than the second diameter.
